(19) United States
(12) Patent Application Publication
(10) Pub. No.: US 2009/0156993 A1
(43) Pub. Date: Jun. 18, 2009

(54) Breast Milk Collection and Storage Device

(76) Inventors: Renee Jetton Callanan, Sheridan, OR (US); Helen J. Anderson, McMinnville, OR (US)

Correspondence Address: PETER A. HAAS, ESQUIRE, 1929 SW 13TH AVENUE, PORTLAND, OR, 97201, US

(21) Appl. No.: 11/956,765

(22) Filed: Dec. 14, 2007

(51) Int. Cl. A61M 1/06; A61M 1/00

(52) U.S. Cl. 604/74; 604/326

(57) ABSTRACT

In a preferred embodiment, the present invention includes a breastmilk collection and storage device that contours to the natural form of a female human breast and consists of an outer layer, an inner layer and a sterile collection bag. Designed to fit under any standard brassiere, the device provides an opening to be comfortably worn around the nipple. The opening directs mild to the sterile collection bag and further provides a sealing means for coupling to a cap or other closure device when not being worn.

### BACKGROUND

The present invention relates to concealable, breastmilk collection and storage devices for lactating mothers.

Natural breastfeeding infants, widely accepted as the best way to nurture an infant, often causes practical limitations particularly to working mothers or otherwise active mothers who are often in public spaces and are unable to immediately locate a private or discrete location where they can comfortably feed their infant or collect the breastmilk for a later use. Recognizing this problem, the prior art presents many varied attempts to provide devices that enable a lactating mother to collect or prevent lactation.

Broadly, the state-of-the-art categorizes into three classifications including concealed devices for collection or storage, non-concealable pumping and storing apparatuses, and concealed lactation prevention devices.

Representative of the first class of concealed devices, devices worn similar to undergarments, includes the concealed apparatus for hands free breast milk pumping and storage of Prentiss disclosed in U.S. Pat. No. 6,440,100 issued on 27 Aug. 2002. The Prentiss apparatus includes a low-profile nipple cap held in place beneath a support brassier. This apparatus permits concealed, hands-free breast pumping in a public environment without any remarkable change in the user's visible appearance. Milk is expressed from the breasts through the nipple cap, which links to a remote reservoir via a milk storage conduit. A vacuum is applied to the storage reservoir by an electronic or manual pump. One limitation of the Prentiss apparatus includes the necessity of an external or remote reservoir bag. Further, the Prentiss apparatus requires a remote or external pump device to extract the milk. The additional, remote or external pump and bag destroy the privacy or operation discretion desired by many mothers, requiring them to find a more private or less-public venue to attend to the collection of breastmilk. Thus, there remains a need for a device that operates when completely concealed, and such a device should further eliminate the extra equipment as taught by Prentiss.

The second category, non-concealable pumping and storing apparatuses, is well represented in the prior art and typically comprise a funnel-like device adapted to fit over the nipple and a portion of a female breast, a conduit member linking the funnel and directing milk to a collection device. Typically, the collection device is a bag. A representative example of the prior art includes a breast milk pump support harness disclosed by Fields in U.S. Pat. No. 6,247,996 issued on 19 Jun. 2001. Fields describes a support harness to be worn by a nursing mother that includes two detachable collection bottle support assemblies and a brassiere assembly. Examples of bags and collection funnels include a disposable sterile bag disclosed by Lafond in U.S. Pat. No. 6,328,082 issued on 11 Dec. 2001. Lafond describes a disposable plastic bag with a liquid receiving chamber having a tear-off strip and closure member. Limitations of this classification of these non-concealable pumping and storing apparatuses include their inability to be concealed beneath outer garments, require remote pumping devices, and are inoperable to discretely collect breastmilk. Thus, there remains a need for a collection system that can easily be worn under outer garments, yet retain a normal appearance for the user when worn. Such a device should include a collection means that does not require a remote or external pump apparatus.

Attempts to both conceal and prevent lactation include devices described by Morrissey et al. in U.S. Pat. No. 5,531,231 issued on 2 Jul. 1996 and in U.S. Pat. No. 5,732,714 issued on 31 Mar. 1998 include an apparatus having an outer surface and an inner surface shaped to conform substantially to a human female breast and having a protrusion with a substantially flat, nipple-contacting surface which extends away from the support and positions to align substantially with and contact a nipple, and thus preventing lactation when placed over the breast. The Morrissey device, however, does not teach or disclose any means for collecting breastmilk, and attempts to prevent lactation. This device does not adequately address the need for a concealed collection device.

Thus, there remains a need for a concealable device that collects breastmilk without requiring an external or remote pump device. Such a device should contour to the natural shape of the female breast to further enhance concealment and comfort during use. Such a device should be easy to wear with a supportive brassiere, provide a normal appearance when viewed under clothing, provide a means for collecting milk that can be reused, easily cleaned, and seal to retain the milk for later use.

### SUMMARY OF THE INVENTION

The present invention overcomes the aforementioned limitations of the prior art and provides a concealable device for collecting breastmilk without requiring use of a remote or external pump device.

In a preferred embodiment, the present invention includes a breastmilk collection and storage device that contours to the natural form of a female human breast and consists of an outer layer, an inner layer and a sterile collection bag. Designed to fit under any standard brassiere, the device provides an opening to be comfortably worn around the nipple. The opening directs mild to the sterile collection bag and further provides a sealing means for coupling to a cap or other closure device when not being worn.

### DRAWING

FIG. 1 is a front view of a first preferred embodiment of the present invention.

FIG. 2 is a back view of the embodiment of FIG. 1.

FIG. 3 is a cross-sectional side view along the line 3-3 of FIG. 2.

FIG. 4 is a partial cross-sectional side view detailing an opening according to a second preferred embodiment of the present invention.

FIG. 5 is a cross-sectional side view along the line 5-5 of FIG. 1.

FIG. 6 is a right side view of a second embodiment of the present invention.

FIG. 7 is a partial cross-sectional side view detailing an opening according to another preferred embodiment of the present invention.

### DESCRIPTION OF THE INVENTION

Possible preferred embodiments will now be described with reference to the drawings and those skilled in the art will understand that alternative configurations and combinations of components may be substituted without subtracting from the invention. Also, in some figures certain components are omitted to more clearly illustrate the invention.

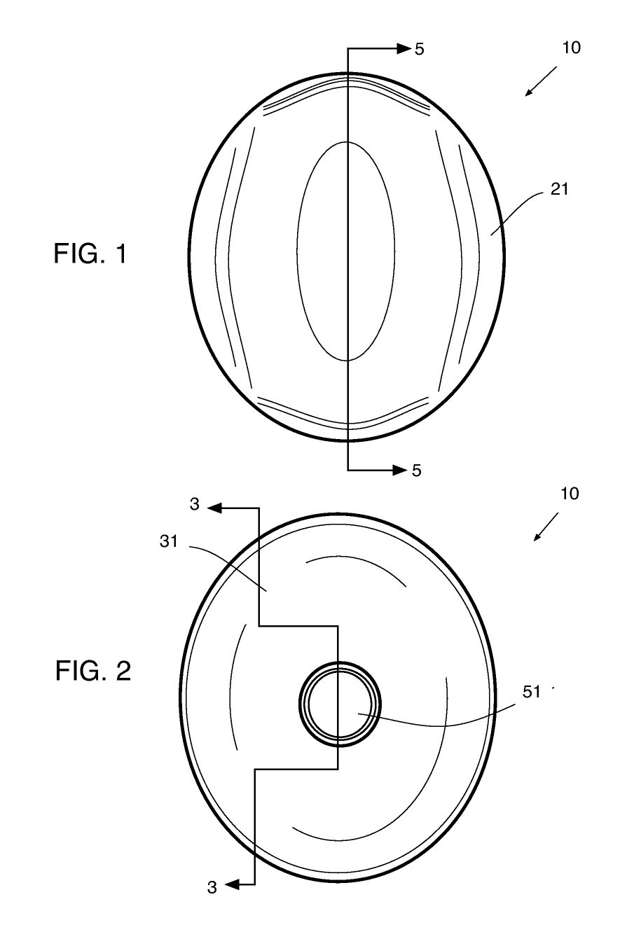

FIGS. 1, 2 and 4 show a first preferred embodiment of the present invention, which consists of a breastmilk collection and storage device 10. The collection and storage device adapts to fit over a human female nipple and portion of a female breast, and preferably under a supportive brassiere to provide a natural shape and appearance when worn in contact with skin under apparel. The device 10 includes an outer layer 21 having a first contour. The first contour emulates the curve and contour of a human female breast. For example the outer layer consists of a generally convex elliptical form when viewed from above and measures about a 6-inch diameter on the minor axis and about an 8-inch diameter on the major axis and consists of a relatively soft and resiliently deformable material such as a silicon-type material as would be appreciated by those with skill in this art. One suitable material for the inner and outer layers includes the brand-name STERalloy FDG available from www.hapcoweb.com, which is a liquid molding polymer alloy that has been specifically designed for food and drug applications.

The device 10 further consists of an inner layer 31 having a second contour. The second contour also emulates the curve and contour of a human female breast, but is sized to create a cavity when coupled to the outer layer. For example, the inner layer 31 consists of a generally concave circular form when viewed from above and measures about 6-inches in diameter.

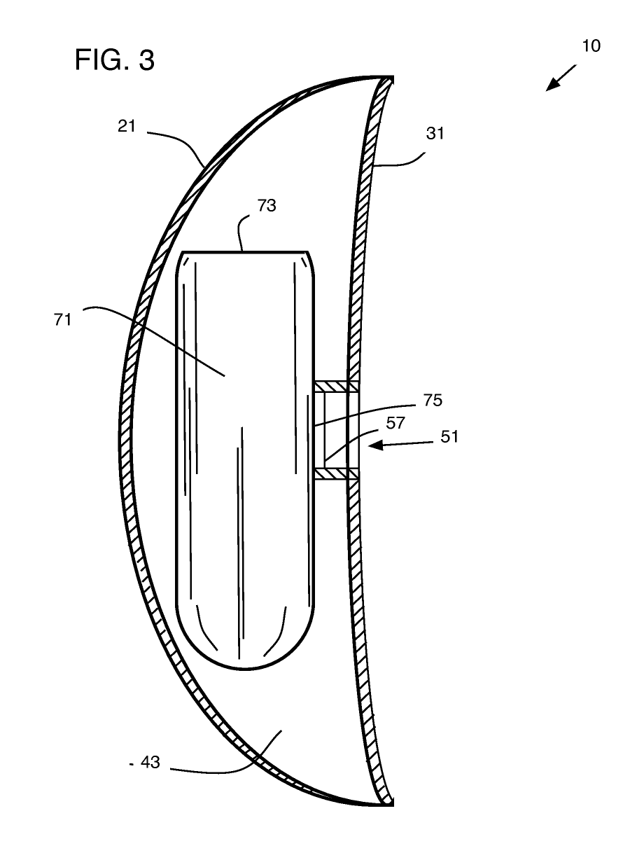

The outer layer 21 couples to the inner layer 31 along two separate portions of a common boundary between the two layers. The two layers align their respective concavity in the same direction to form a cavity 43, a volume that appears generally crescent-shaped when viewed in profile. FIG. 3 clearly illustrates the crescent-shaped cavity 43 with respect to the inner layer 31 and outer layer 21. As would be understood in this art, a portion of the boundary between the outer layer and inner layer aligns coincident with each other, and serves as a join-edge during the fabrication process. This join-edge may readily be adhered by using a suitable adhesive or molded in the desired configuration. Also, a portion of the boundary between the inner and outer layers will remain open and not coupled together. This opening lends itself readily to aiding the insertion and retraction of the collection bag 41.

In certain alternative preferred embodiments, the join-edge consists of a portion of the common boundary between the inner and outer layers and acts as a hinge. In this way the inner layer may nest in close proximity to the outerlayer for storage or when worn. Thus, as breastmilk collects in the collection bag, the outer layer may expand outward with respect to the wearer and the cavity 43 would vary in volume as the outer layer adjusts to changes in the volume of milk collected.

Facilitating collection of breastmilk, the device 10 further includes a nipple-receiving opening 51 disposed on the inner layer 31. The opening 51 adapts to enable the human female nipple to insert therethrough and create a fluid conduit from the inner layer to the cavity 43 or directly to a collection bag 41.

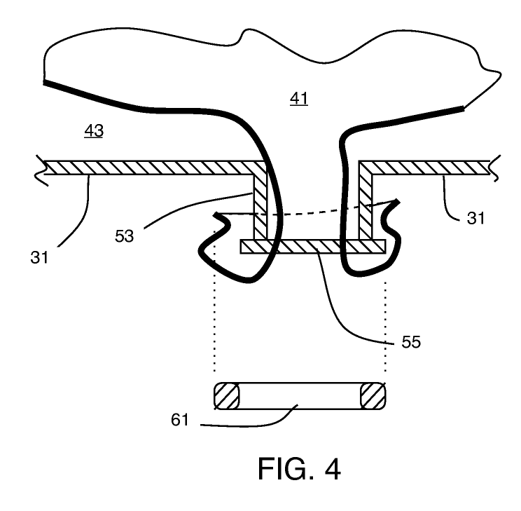

FIG. 4 more clearly illustrates a suitable collection bag 41, which adapts to arrange within the cavity 43 formed between the inner and outer layers. The cavity may include several openings, as previously discussed. The collection bag 41—any number of disposable plastic and sterile bags adapted for use to collect breastmilk, as would be well understood in this art, can adapt for use as the collection bag of the present invention—arranges in the cavity and pesents an opening to align with the nipple-receiving opening 51 so that breastmilk can flow from the lactating mother's breast into the collection bag. The collection bag, accordingly, selectively couples to the inner layer, or to the opening 51.

FIG. 4 also shows one alternative preferred embodiment of a suitable nipple-receiving opening 51. This opening is circular (as FIG. 2 illustrates) in cross section—although other shapes can work equally well including a slit, oval, or rectangle, for example. In this embodiment, the opening 51 includes a shoulder 53 protruding from the surface of the inner layer 31 at a first shoulder end. A circular flange 55 forms at an oppositely disposed second shoulder end. The flange 55 cooperates with the retaining member 61, for example an elastically deformable member such as an O-ring or rubber band, to retain an open end of the collection bag 41 in position to enable fluid flow into the collection bag.

The retention member 61, in another preferred embodiment, removes from the nipple-receiving opening 51, enabling the now-full collection bag to be removed from the inner layer and outer layer assembly. And, the retention member 61 now serves as a sealing member to close the now-full collection bag to prevent leakage.

FIG. 3 shows another preferred embodiment of a breastmilk collection device 10 having a rigid inner cup 71 formed or molded from a polyproponal or other similar plastic material. The inner cup includes an open top portion 73 and vertical sidewalls and a bottom cooperating to form a compartment 77. An access port 75 disposed on one vertical sidewall aligns with the fluid conduit 57 portion of the inner layer 31 to provide a fluid-flow path from the female nipple to the collection cup 71.

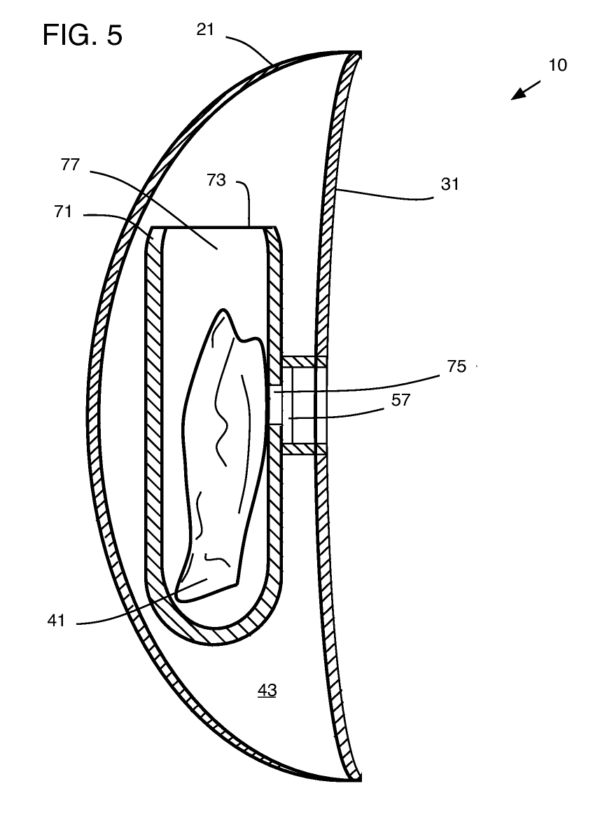

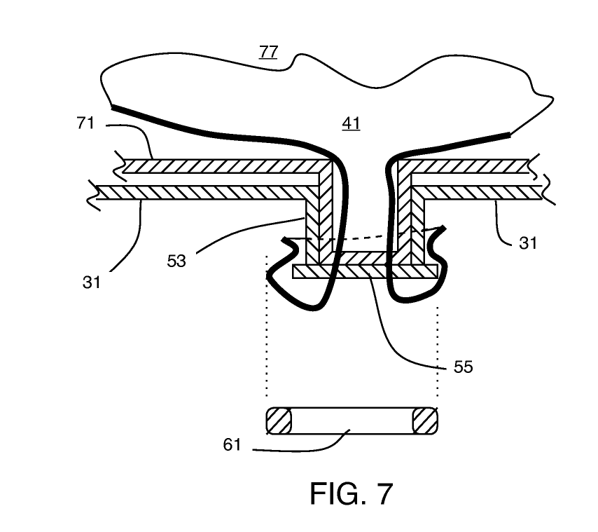

FIG. 5 shows the collection cup 71 being used with a bag liner 41, which can easily be inserted through the open top 73. FIG. 7 further details a possible arrangement of components relative to the inner layer 31. An o-ring type sealing member 61 or rubber band inserts over a flange 55 onto a neck 53 of the fluid conduit 57, which provides a fluid passage to the inner compartment 77 of the cup 71 or to a bag liner 41 inserted in the same inner compartment 77.

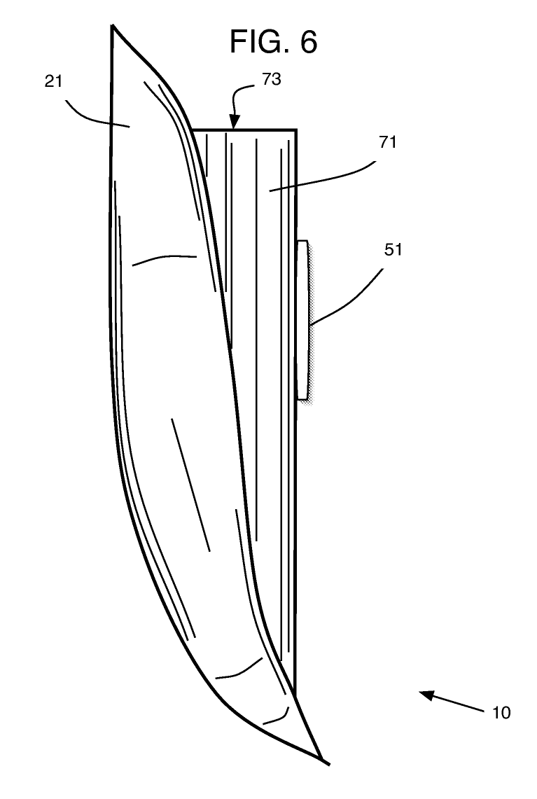

FIG. 6 shows another preferred embodiment of the present invention. An outer layer 21 and cooperating inner layer 31 (not shown in FIG. 6) sandwiches an inner cup 71 with an open top 73. A port 51 enables breastmilk to flow into the compartment of the cup 71.

Although the invention has been particularly shown and described with reference to certain embodiments, it will be understood by those skilled in the art that various changes in form and detail may be made without departing from the spirit and scope of the invention.

We claim:

1. A breastmilk collection and storage device comprising:
an outer layer having a first contour;
an inner layer having a second contour and coupled to the outer layer to form a cavity and
a nipple-receiving opening creating a fluid conduit from the inner layer to the cavity.

2. The device of claim 1 further comprising:
a means for collecting breastmilk coupled to the nipple-recieiving opening and disposed in the cavity, the means comprising a bag liner.

3. The device of claim 1 further comprising:
an inner cup container adapted to receive breastmilk;
the cup container having at least one vertical sidewall, a bottom and an open top, the sidewall, bottom and top cooperating to form a compartment adapted to hold breastmilk; and
the vertical sidewall further comprising a port adapted to cooperate with the nipple-receiving opening whereby a fluid conduit is formed from the inner layer to the inner compartment of the cup container.

4. A breastmilk collection and storage device adapted to fit over a human female nipple and portion of a female breast, the device comprising:
an outer layer having a first contour;
an inner layer having a second contour and coupled to the outer layer to form a cavity;
a nipple-receiving opening adapted to enable the human female nipple to insert therethrough, the nipple-receiving opening creating a fluid conduit from the inner layer to the cavity;
a collection bag disposed in the cavity, the bag having an open end adapted to couple to the nipple-receiving opening; and
a retaining means for selectively coupling the bag open end to the nipple-receiving opening.

5. The breastmilk collection and storage device of claim 4 wherein the retaining means further comprises a sealing means for closing the collection bag when removed from the cavity.

6. The breastmilk collection and storage device of claim 4 wherein the retaining means further comprises an elastically deformable retention member.

7. The breastmilk collection and storage device of claim 4 wherein:
the nipple-recieiving opening further comprises a shoulder coupled to the inner layer at a shoulder first end, and a flange disposed at a should second end, the flange adapted to selectively receive the retaining means; and
the retaining means further comprises an elastically deformable retention member.
